(19) United States
(12) Patent Application Publication
(10) Pub. No.: US 2025/0110023 A1
(43) Pub. Date: Apr. 3, 2025

(54) SYSTEMS AND METHODS FOR INTEGRATED AUDIO-OPTICAL TESTING

(71) Applicant: Scott Lineweaver, Chandler, AZ (US)

(72) Inventor: Scott Lineweaver, Chandler, AZ (US)

(21) Appl. No.: 18/904,411

(22) Filed: Oct. 2, 2024

Related U.S. Application Data
(60) Provisional application No. 63/587,155, filed on Oct. 2, 2023.

(51) Int. Cl. G01M 99/00; A61B 3/00

(52) U.S. Cl. CPC G01M 99/005; A61B 3/00

(57) ABSTRACT

A diagnostic test assembly for an ophthalmic device comprising a housing, an audio output module disposed within the housing, a tone generator module disposed within the housing and in electronic communication with the audio output module, a user interface module disposed within the housing, a mechanical interface module integrated with the housing and configured to removably couple the housing to the ophthalmic device, a processor disposed within the housing and in electronic communication with the user interface module and the tone generator module, a tangible, non-transitory electronic memory in communication with the processor, the tangible, non-transitory memory having instructions stored thereon that, in response to execution by the processor, cause the processor to perform operations.

### CROSS-REFERENCE TO RELATED APPLICATIONS

This application is a non-provisional of, and claims priority to, U.S. Provisional Application No. 63/587,155, Oct. 2, 2023, entitled “HEARING TEST AND EYE REFLEX DEVICE FOR OPTICAL PROFESSIONALS” and which is hereby incorporated by reference in its entirety.

### FIELD

The disclosure relates generally to apparatus, systems, and methods for analysis,

diagnosing, monitoring, and/or treating audio and visual disorders and, more particularly, to integrated ophthalmic diagnostic devices.

### BACKGROUND

Ophthalmic instruments and techniques are routinely used by clinicians to diagnose and treat eye-related ailments. The patient may be positioned in a specific, seated position for the entire duration of the procedure, which may last anywhere between a few seconds to a few minutes. This positioning has been considered necessary to properly align the patient's eye with the ophthalmic device, to perform measurements and/or therapeutic procedures on the patient's eyes.

### SUMMARY

In various embodiments the present disclosure provides a diagnostic test assembly for an ophthalmic device, comprising a housing, an audio output module disposed within the housing, a tone generator module disposed within the housing and in electronic communication with the audio output module, a user interface module disposed within the housing, a mechanical interface module integrated with the housing and configured to removably couple the housing to the ophthalmic device, a processor disposed within the housing and in electronic communication with the user interface module and the tone generator module, a tangible, non-transitory electronic memory in communication with the processor, the tangible, non-transitory memory having instructions stored thereon that, in response to execution by the processor, cause the processor to perform operations comprising receiving, by the processor, a start test command from the user interface module, executing, by the processor, the tone generator module in response to receiving the start test command, wherein, in response to execution by the processor, the tone generator module generates a discrete series of pure tone sine waves and feeds the discrete series of pure tone sine waves to the audio output module for further processing, wherein, in response to receiving the discrete series of pure tone sine waves, the audio output module modulates the output level of each tone of the of the discrete series of pure tone sine waves across a discrete series of audio output levels.

In various embodiments, the discrete series of pure tone sine waves consists of tones at 500 Hz, 1000 Hz, 2000 Hz, and 4000 Hz. In various embodiments, the modulation of the audio output level begins at an output level of 50 dB and decreases in intensity thereafter to 0 dB. In various embodiments, the modulation of the audio output level decreases by decrements of 5 dB.

In various embodiments, the user interface module comprises at least one of a button or a light emitting diode. In various embodiments, the mechanical interface module comprises clamp portion and a ball-and-socket joint, wherein the ball-and-socket joint couples the clamp portion to the housing and enables rotation of the housing relative to the clamp portion at the ball-and-socket joint. In various embodiments, a ball portion of the ball-and-socket joint is disposed within the housing and retained therein by a retaining plug.

In various embodiments, the mechanical interface module further comprises a hook portion and a clamp portion, wherein the hook portion removably couples with a forehead rest of the ophthalmic device and the clamp portion removably couples with an upright member of the ophthalmic device.

In various embodiments, the mechanical interface module further comprises a forehead rest coupled to the housing and a spring coupled between the housing and the mechanical interface module. In various embodiments, the mechanical interface module coupled to an upright member of the ophthalmic device, to in response to a force applied at an inward face of the forehead rest, the housing rotates about the upright member from a first position to a second position and generates a spring force via the spring. In various embodiments, in response to removing the force applied at the inward face of the forehead rest, the spring force acts to rotate the housing from the second position to the first position.

In various embodiments, the mechanical interface module further comprises a first fastener and a second fastener, wherein each of the first fastener and the second fastener are disposed through a forehead rest of the ophthalmic device, the mechanical interface module, and into an upright member of the ophthalmic device to enable coupling therebetween. In various embodiments, the mechanical interface module further comprises a clamp portion coupled to the housing via an angled pivot, wherein the housing is enabled to rotate freely about the pivot, and wherein with the clamp portion coupled to an upright member of the ophthalmic device, in response to a gravitational force action on the housing, the housing rotates to rest at a low side of the pivot.

In various embodiments, the mechanical interface module further comprises a cam ring having a beveled face contacting a portion of the housing such that when coupled about an upright member of the ophthalmic device a rotation of the housing relative to the cam ring causes axial displacement of the housing along the upright member relatively away from the cam ring.

In various embodiments, the present disclosure provides a diagnostic test assembly for an ophthalmic device, comprising a mobile device including a display and an audio output device, a mechanical interface module configured to removably couple the mobile device to the ophthalmic device, a processor, a tangible, non-transitory electronic memory in electronic communication with the processor, and a set of computer readable code on the non-transitory electronic memory, including a user interface (UI) module executable to display system information and receive user inputs, a tone generator module executable by the processor to generate pure tone sine waves between 20 Hz and 10,000 Hz, an audio output module executable by the processor to generate audio output via the audio output device., wherein, in response to execution by the processor, the UI module awaits to receive a start test command, wherein, in response to receiving the start test command, the UI module calls the processor to execute the tone generator module, wherein, in response to execution by the processor, the tone generator module generates a discrete series of pure tone sine waves and feeds the discrete series of pure tone sine waves to the audio output module for further processing, wherein, in response to receiving the discrete series of pure tone sine waves, the audio output module controls the audio output device to output, sequentially, each tone of the of the discrete series of pure tone sine waves across a discrete series of audio output levels.

In various embodiments, the test assembly further comprises a native application executable on the mobile device, wherein the native application includes the UI module, the tone generator module, and the audio output module.

In various embodiments, the discrete series of pure tone sine waves consists of tones at 500 Hz, 1000 Hz, 2000 Hz, and 4000 Hz. In various embodiments, the discrete series of audio output levels begins at an output level of 50 dB and decreases in intensity thereafter to 10 dB or less. In various embodiments, wherein the discrete series of audio output levels steps down by decrements of at least 5 dB to 10 dB. In various embodiments, the mechanical interface module includes a clamp portion, a mobile device mounting portion, and an arm portion, wherein the clamp portion and the mobile device mounting portion are coupled at distal ends of the arm.

The forgoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

The subject matter of the present disclosure is particularly pointed out and distinctly claimed in the concluding portion of the specification. A more complete understanding of the present disclosures, however, may best be obtained by referring to the detailed description and claims when considered in connection with the drawing figures, wherein like numerals denote like elements.

FIG. 1 illustrates a diagnostic test assembly for an ophthalmic device, in accordance with various embodiments;

FIG. 2 illustrates an ophthalmic device having a diagnostic test assembly, in accordance with various embodiments;

FIG. 3 illustrates a mechanical interface module of a diagnostic test assembly, in accordance with various embodiments;

FIG. 4 illustrates a block diagram of mobile device of a diagnostic test assembly, in accordance with various embodiments;

FIG. 5 illustrates a slit lamp assembly including a diagnostic test assembly, in accordance with various embodiments;

FIG. 6 illustrates a block diagram of a diagnostic test assembly, in accordance with various embodiments;

FIG. 7 illustrates a mechanical interface module of a diagnostic test assembly, in accordance with various embodiments;

FIG. 8 illustrates a housing and an integrated mechanical interface module of a diagnostic test assembly, in accordance with various embodiments;

FIG. 9 illustrates a housing and an integrated mechanical interface module of a diagnostic test assembly, in accordance with various embodiments;

FIG. 10A illustrates mechanical interface module of a diagnostic test assembly, in accordance with various embodiments;

FIG. 10B illustrates mechanical interface module of a diagnostic test assembly, in accordance with various embodiments;

FIG. 11 illustrates mechanical interface module of a diagnostic test assembly, in accordance with various embodiments;

FIGS. 12A, 12B, and 12C illustrate a mechanical interface module of a diagnostic test assembly, in accordance with various embodiments;

FIG. 13 illustrates mechanical interface module of a diagnostic test assembly, in accordance with various embodiments;

FIGS. 14A and 14B illustrate a cam ring assembly of a mechanical interface module, in accordance with various embodiments;

FIG. 15 illustrates a diagnostic test assembly, in accordance with various embodiments;

FIG. 16 illustrates a diagnostic test assembly, in accordance with various embodiments; and

FIG. 17 illustrates a diagnostic test assembly, in accordance with various embodiments.

### DETAILED DESCRIPTION

The detailed description of exemplary embodiments herein makes reference to the accompanying drawings, which show exemplary embodiments by way of illustration and their best mode. While these exemplary embodiments are described in sufficient detail to enable those skilled in the art to practice the disclosures, it should be understood that other embodiments may be realized and that logical, chemical, and mechanical changes may be made without departing from the spirit and scope of the disclosures. Thus, the detailed description herein is presented for purposes of illustration only and not of limitation. For example, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Over-the-counter (OTC) hearing aids have altered the landscape of hearing care by providing patients direct access to affordable non-prescription devices. Traditional methods of audiology screening involves bringing a patient to purpose built audiological examination room such as, for example, a sound booth to perform extensive testing over the entire range of hearing. However, for the range of treatment offered by OTC hearing aids such involved and time-consuming tests and purpose-built examination rooms may tend to be overly burdensome on medical resources and time spent by doctors and patients during examination. The systems and methods of the present disclosure present and advancement over traditional methods by enabling audiological screening of a patient while performing ophthalmological examination in a standard ophthalmology examination room. In this regard, the present disclosure provides improvements over traditional systems by reducing the required resources for examination and by reducing the time spent in examination by doctors and patients.

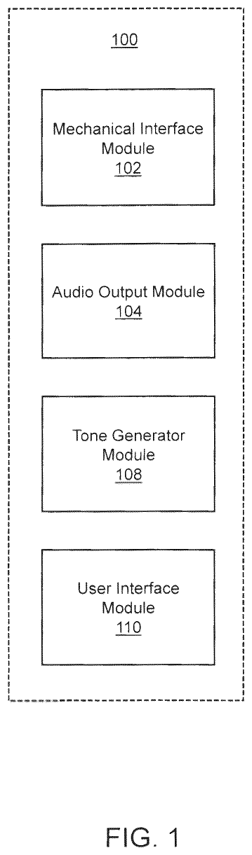

In various embodiments and with reference to FIG. 1, a diagnostic test assembly 100 for an ophthalmic device is illustrated. Diagnostic test assembly 100 comprises a mechanical interface module 102, an audio output module 104, a tone generator module 108 and a User Interface (UI) module 110. The mechanical interface module 102 facilitates mechanical coupling of the audio output module 104, tone generator module 108 and UI module 110 to an ophthalmic device such as, for example, a slit lamp assembly or ophthalmic workstation. Each of the audio output module 104, tone generator module 108 and UI module 110 are in electronic communication. The audio output module 104, tone generator module 108 and user interface module 110 may be computer based, and may comprise one or more processors, a tangible non-transitory computer-readable memory, and/or a network interface along with other suitable system software and hardware components. The one or more processors can be a general purpose processor, a microprocessor, a microcontroller, a digital signal processor (DSP), an application specific integrated circuit (ASIC), a field programmable gate array (FPGA) or other programmable logic device, discrete gate or transistor logic, discrete hardware components, or any combination thereof.

In various embodiments, the UI module 110 enables receiving commands from and displaying information to a user of the diagnostic test assembly 100. For example, the user interface may include a touch screen, button, keypad, light emitting diode, or electronic display. A user may interact with a button or other input device of the UI module to generate a start test command.

In various embodiments, the tone generator module 108 generates pure tone sine waves between 20 Hz and 20,000 Hz. For example, the tone generator module 108 may generate a discrete series of pure tone sine waves consisting of tones at 500 Hz, 1000 Hz, 2000 Hz, and 4000 Hz. The tone generator module may output the pure tone sine waves to the audio output module 104 for further processing. In response to receiving one or more pure tone sine waves from the tone generator module 108, the audio output module 104 modulates the output level of the pure tone sine wave across at least three audio levels. The audio output module 104 may modulate the output level of each tone of the of the discrete series of pure tone sine waves across a discrete series of audio output levels. For example, the modulation of the audio output level may be a discrete series beginning at an output level of 50 dB and decreasing in intensity thereafter to 10 dB or less in decrements of at least 5 dB to 10 dB. In various embodiments, the audio output module 108 may generate a burst or pulse of a very loud sound of about 100 dB where about in this context means ±10 dB. The sound pulse may be a pure tone, white noise, a pop, a snap, a click, a thump, or the like.

In this way, the diagnostic test assembly 100 may step each tone of the discrete series of pure tone sine waves through each of the discreet series of audio output levels. For example, the audio output module 104 may first output the 4000 Hz tone at 50 dB, then 40 dB, then 30 dB, then 10 dB, thereafter outputting the 2000 Hz tone at 50 dB, then 40 dB, then 30 dB, then 10 dB, and so forth stepping through the tones. Patient hearing loss from 0-25 dB across discrete series of pure tone sine waves [500 Hz, 1 kHz, 2 kHz, 4 kHz) may be determined as normal to slight loss and no further testing or treatment may be ordered. Patient hearing losses in one or more of aforementioned test frequencies from 26 dB to 50 dB may be determined to be mild to moderate loss and OTC or prescription hearing aids may be recommended. Patient hearing losses in one or more frequencies of aforementioned test frequencies greater than 50 dB may be determined as a category of loss requiring prescriptive hearing aids and a referral to an audiologist or other specialist such as, for example, an ENT practitioner.

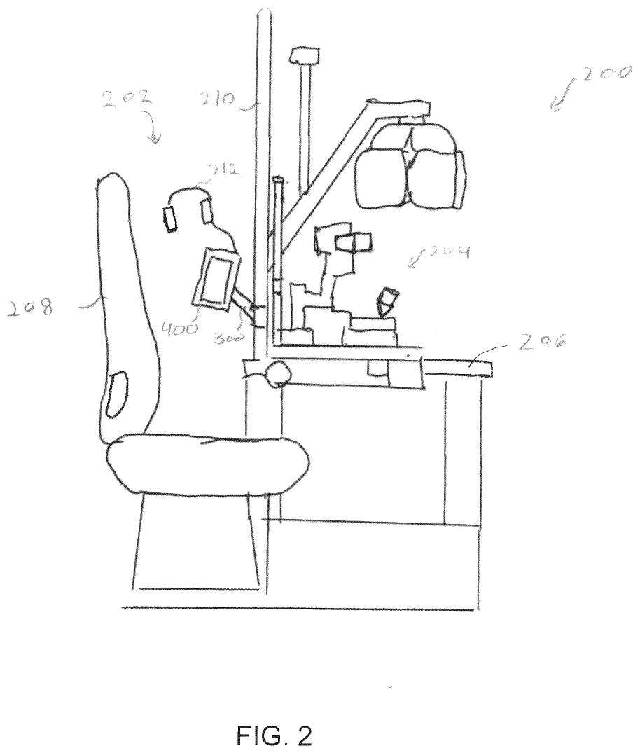

With additional reference to FIG. 2, an ophthalmic device 200 is illustrated coupled with a diagnostic test assembly 202 in accordance with various embodiments. The diagnostic test assembly 202 may comprise various features, materials, and components similar to those of diagnostic test assembly 100 and includes a mobile device 400 and a mechanical interface module 300. The mechanical interface module 300 removably couples the mobile device 400 to an upright member 210 of the ophthalmic device 200. In various embodiments, the ophthalmic device may comprise a slit lamp assembly 204 and/or an ophthalmic workstation 206 which may provide support to the slit lamp assembly 206 and/or other ophthalmic devices or instruments. The ophthalmic workstation 206 may include an examination chair 208 configured to support the patient while under examination by one or more of the ophthalmic instruments at the workstation 206. The mechanical interface module 300 is configurable to position the mobile device 400 within ergonomic reach of a patient seated in the examination chair 208. In various embodiments, headphones 212, speakers, earbuds, and or the like may be coupled to or wirelessly communicate with the mobile device 400 enabling the patient to hear sounds produced on the mobile device 400.

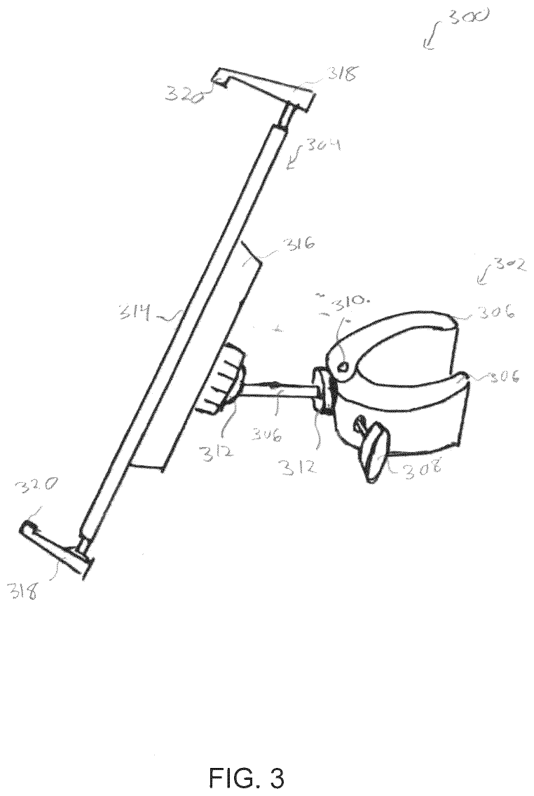

With additional reference to FIG. 3, a mechanical interface module 300 is illustrated in accordance with various embodiments. In various embodiments, mechanical interface module 300 includes a clamp portion 302, a mobile device mounting portion 304, and an arm portion 306. The clamp portion 302 and the mobile device mounting portion 304 are coupled at distal ends 312 of the arm portion 306. In various embodiments the distal ends 312 may include articulating joints such as, for example, a ball joint which enables movement or positioning of the arm position 306 and the mobile device mounting portion 304 relative to the clamp portion 302.

In various embodiments, the clamp portion 302 comprises jaws 306 which are curved or contoured generally to the shape of the upright member 210 of the ophthalmic device 200. Jaws 306 may be coupled via a hand screw 308 and a pivot 310 such that rotation of the hand screw 308 may cause the jaws 306 to open or close and thereby enable coupling of the clamp portion 302 to the ophthalmic device.

In various embodiments, the mobile device mounting portion 304 may include a rare earth permanent magnet or other magnetic coupling sufficient to cause the mobile device 400 to adhere securely at face 314. The mobile device mounting portion 304 may further comprise an adjustable clamp mechanism 316 which enables jaws 318 to couple the mobile device 400 to the face 314. For example, mobile device 400 may have a cuboid shape and the jaws 318 and clamp mechanism 316 may couple the mobile device to the face 314 by generating an interference at opposing edges of the mobile device 400 or at the corners of the mobile device 400. In various embodiments, jaws 318 may include a flanged portion 320 which may intrude over an outward facing portion of the mobile device 400 and thereby tend to inhibit removal of the mobile device when the device is pulled directly away from the face 314.

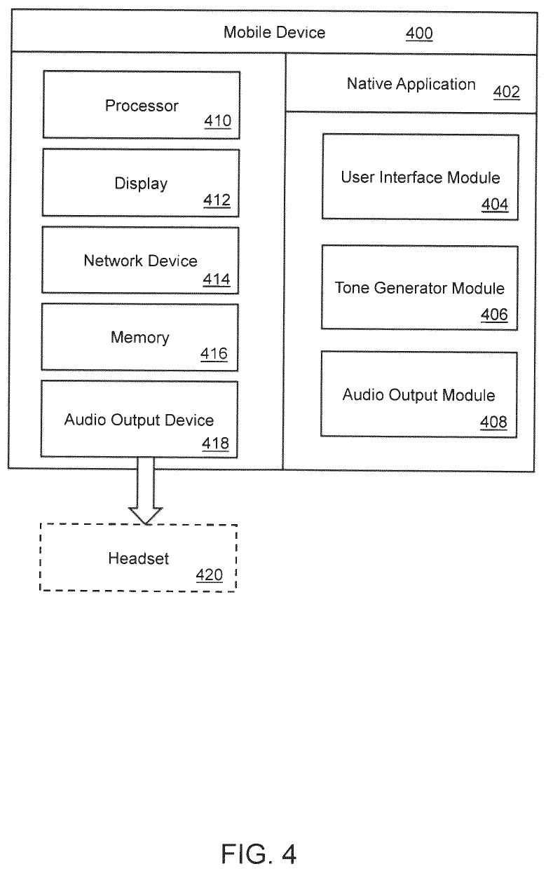

In various embodiments and with additional reference to FIG. 4, a block diagram of mobile device 400 is illustrated. The mobile device 400 may comprise software and/or hardware in communication with the system modules. In various embodiments, mobile device 400 includes a processor 410, a display 412, a network device 414, memory 416, and an audio output device 418. In various embodiments, the processor 410 is configured as a central network element or hub facilitating electronic communication between all the system elements and components of mobile device 400. It should be appreciated that mobile device 400 is only one example of a mobile device and that it may have more or fewer components than shown or a different configuration of components. The various components shown may be implemented in hardware, software or a combination of both hardware and software including one or more signal processing and/or ASICs. The mobile device 400 may comprise any suitable device that is configured to allow a user to communicate with a network and/or the system modules. The mobile device 400 may include, for example, a personal computer, smartphone, tablet, and/or the like and may allow a user to transmit data and messages to the system and to receive feedback from the system.

In various embodiments, memory 416 may include high speed random access memory and may also include non-volatile memory, such as one or more magnetic memory devices, flash memory devices, or other non-volatile solid state memory devices. In some embodiments, memory 416 may include storage remotely located from the processor 410, for instance network storage accessible via the network device 414 and a communications network (not shown) such as the internet, SANs, WANs, LANs, WLANs and/or the like.

The mobile device 400 may include a network device 414 which enables electronic communication with a network (not shown) or other system components. A native application 402 may be installed on the memory 416 and executable by the processor 410 of the mobile device 400 via download, physical media, or a platform specific app store, for example. The native application 402 may utilize the development code base provided for use with the operating system of the particular mobile device. The native application 402 may be configured to perform system calls and to manipulate the stored and displayed data on the mobile device 400. The native application 402 may include a user interface module 404, a tone generator module 406, and an audio output module 408. In various embodiments, the native application 402 may be configured to communicate with an application server (e.g., via network device 414 and a network) and to process data received from the application server (not shown).

In various embodiments, display 412 may be a touch screen which provides both an output interface and an input interface between the mobile device 400 and the user (e.g., the patient). Display 412 may provide electrical signals to the processor in response to haptic and/or tactile interactions from the user. Display 412 may provide visual output to the user including text, graphics, video, and any combination thereof. The user interface module 404 of the native application 402 may be in electronic communication with the display 412 and enable the native application 402 to receive data from the user and to display data to the user. In this regard, the user interface module is executable by the processor 410 to display system information and receive user inputs. For example, in response to execution by the processor 410, the user interface module 404 may awaits to receive a start test command from the processor. The start test command may be generated in response to a haptic and/or tactile interaction with a soft button displayed on the display 412 by the user interface module 404.

In various embodiments, audio output device 418 provides an audio interface between the mobile device 400 and the user. The audio output device 418 receives audio data from the audio output module 408 of the native application 402 and converts the audio data into an electric signal for transmission to a peripheral device such as a headset 420, a speaker, and/or the like. In various embodiments, the audio output device 418 may convert the audio data into an RF signal for transmission to a wireless audio peripheral.

In various embodiments, the tone generator module 406 is executable by the processor 410 to generate pure tone sine waves between 20 Hz and 10,000 Hz. In response to receiving the start test command, the user interface module 404 may call the processor 410 to execute the tone generator module 406. In response to execution by the processor 410, the tone generator module 406 generates a discrete series of pure tone sine waves and feeds the discrete series of pure tone sine waves to the audio output module 408 for further processing.

In various embodiments, the audio output module 408 is executable by the processor 410 to generate audio output via the audio output device 418. In response to receiving the discrete series of pure tone sine waves, the audio output module 408 controls the audio output device 418 to output, sequentially, each tone of the of the discrete series of pure tone sine waves across a discrete series of audio output levels. For example, the audio output module 408 may pass audio data comprising the discrete series of pure tone sine waves and the discrete series of audio output levels to the audio output device 418 for conversion and transmission to an audio peripheral such as a speaker or headset. In various embodiments, the audio output module 108 controls the audio output device 418 to output, sequentially, each tone of the of the discrete series of pure tone sine waves across a discrete series of audio output levels. For example, where the discrete series of pure tone sine waves comprises [500 Hz, 1000 Hz, 2000 Hz, 4000 Hz] and where the discrete series of audio output levels comprises [50 dB, 40 dB, 30 dB, 10 dB] the audio output module 408 may control the audio output device 418 to first output the 4000 Hz tone at 50 dB, then 40 dB, then 30 dB, then 10 dB, thereafter outputting the 2000 Hz tone at 50 dB, then 40 dB, then 30 dB, then 10 dB, and so forth stepping through the tones and output levels.

In various embodiments, in response to a reflex test command from the processor 410, the audio output module 408 may generate a reflex test sound. The reflex test sound comprises burst and/or pulse of a very loud sound of about 100 dB where about in this context means ±10 dB. Audio output module 408 may pass audio data comprising the reflex test sound to the audio output device 418 to output for conversion and transmission to an audio peripheral. A practitioner may observe the user's eyes via the ophthalmic device for an eye reflex responsive to the reflex test sound and record the response time which may be employed diagnostically for assessment of neurological damage, cognitive decline, and or the like.

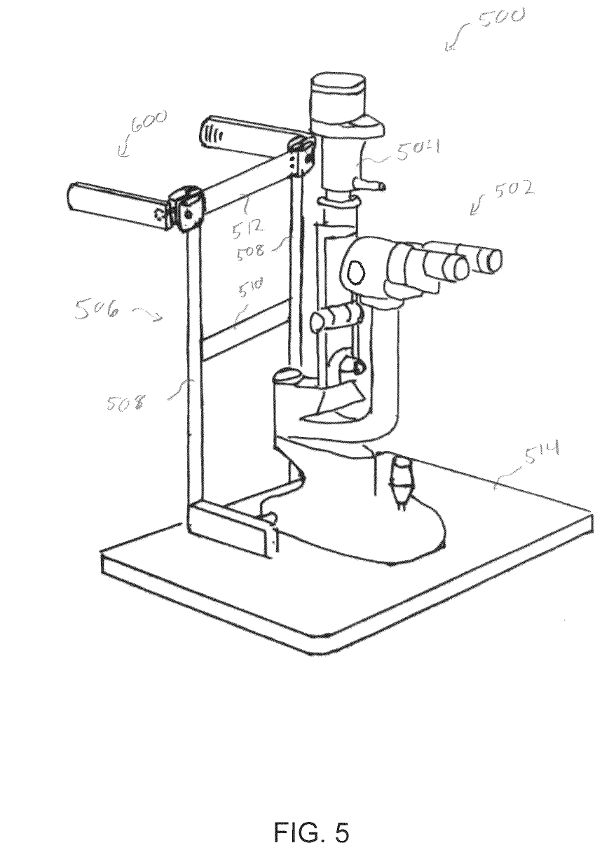

With additional reference to FIG. 5, a slit lamp assembly 500 is illustrated in accordance with various embodiments. Slit lamp assembly 500 includes a slit lamp microscope 502, an illumination system 504, a head fixture 506, and a support table 514. The slit lamp microscope 502 and head fixture 506 may be coupled to a support table 514. The head fixture 506 is configured to align the patients head with slit lamp microscope 502 for examination. In various embodiments the head fixture 506 includes a pair of upright members 508, a chin rest 510, and a forehead rest 512. The chin rest is coupled between the upright members 508 and may be adjustable relatively upward or downward from the support table 514 along the length of the upright member 508. The forehead rest 512 may be coupled between the upright member 508 at an end of the upright members distal of the support table 514. In various embodiments, a diagnostic test assembly 600 is coupled to the upright member 508 proximate the forehead rest 512.

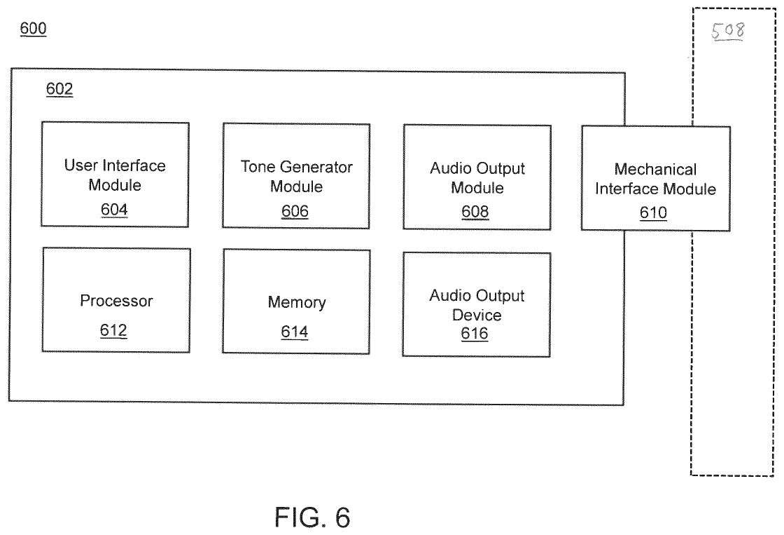

With additional reference to FIG. 6, a block diagram of a diagnostic test assembly 600 is illustrated in accordance with various embodiments. Diagnostic test assembly 600 may comprise various features, materials, and components similar to those of diagnostic test assembly 100 and 202 but includes a housing 602 which is integrated in common with the hardware components of the diagnostic test assembly 600. In various embodiments, each of the user interface module 604, tone generator module 606, audio output module 608, processor 612, memory 614, and audio output device 616 are disposed within the housing 602. Mechanical interface module 610 is integrated with the housing 602 to removably couple the housing 602 to an upright member of an ophthalmic device such as, for example, upright member 508 of slit lamp assembly 500. In various embodiments, portions of the mechanical interface module 610 may be monolithic with the housing 602.

In various embodiments, processor 612 may comprise one or more processors, a tangible non-transitory computer-readable memory (such as memory 614), and/or a network interface along with other suitable system software and hardware components. The one or more processors can be a general purpose processor, a microprocessor, a microcontroller, a digital signal processor (DSP), an application specific integrated circuit (ASIC), a field programmable gate array (FPGA) or other programmable logic device, discrete gate or transistor logic, discrete hardware components, or any combination thereof.

In various embodiments, memory 614 may include high speed random access memory and may also include non-volatile memory, such as one or more magnetic memory devices, flash memory devices, or other non-volatile solid state memory devices. In some embodiments, memory 416 may include storage remotely located from the processor 410, for instance network storage accessible via a network device (not shown) and a communications network (not shown) such as the internet, SANs, WANs, LANs, WLANs and/or the like.

In various embodiments, user interface module 604 may provide electrical signals to the processor 612 in response to haptic and/or tactile interactions from the user and thereby enables receiving commands from the user. The user interface module 604 may display information from the system to the user. For example, the user interface module 604 may include a touch screen, button, keypad, light emitting diode, or other electronic display. A user may interact with a physical button or other input device of the user interface module 604 to generate a start test command. The user interface module 604 may transmit the start test command to the processor 612 for further processing.

In various embodiments, the tone generator module 606 is executable by the processor 612 to generate pure tone sine waves between 20 Hz and 10,000 Hz. In response to receiving the start test command, the processor 612 executes the tone generator module 606. In response to execution by the processor 612, the tone generator module 606 generates a discrete series of pure tone sine waves and feeds the discrete series of pure tone sine waves to the audio output module 608 for further processing.

In various embodiments, the audio output module 608 generates audio output data. In various embodiments, in response to receiving the discrete series of pure tone sine waves the audio output module 608 modulates the output level of each tone of the of the discrete series of pure tone sine waves across a discrete series of audio output levels. For example, in response to receiving the discrete series of pure tone sine waves, the audio output module 608 may control the audio output device 616 to output, sequentially, each tone of the of the discrete series of pure tone sine waves across a discrete series of audio output levels. For example, the audio output module 608 may pass audio data comprising the discrete series of pure tone sine waves and the discrete series of audio output levels to the audio output device 616 for conversion and playback via as a speaker (not shown) integrated within the housing 602. In various embodiments, the audio output module 608 controls the audio output device 616 to output, sequentially, each tone of the of the discrete series of pure tone sine waves across a discrete series of audio output levels. For example, where the discrete series of pure tone sine waves comprises [500 Hz, 1000 Hz, 2000 Hz, 4000 Hz] and where the discrete series of audio output levels comprises [50 dB, 40 dB, 30 dB, 10 dB] the audio output module 408 may control the audio output device 418 to first output the 4000 Hz tone at 50 dB, then 40 dB, then 30 dB, then 10 dB, thereafter outputting the 2000 Hz tone at 50 dB, then 40 dB, then 30 dB, then 10 dB, and so forth stepping through the tones and output levels.

In various embodiments, in response to a reflex test command from the processor 612, the audio output module 608 may generate a reflex test sound. The reflex test sound comprises burst and/or pulse of a very loud sound of about 100 dB where about in this context means ±10 dB. Audio output module 608 may pass audio data comprising the reflex test sound to the audio output device 616 for conversion and playback via as a speaker (not shown) integrated within the housing 602. A practitioner may observe the user's eyes via the slit lamp microscope 502 for an eye reflex responsive to the reflex test sound and record the response time which may be employed diagnostically for assessment of neurological damage, cognitive decline, and or the like.

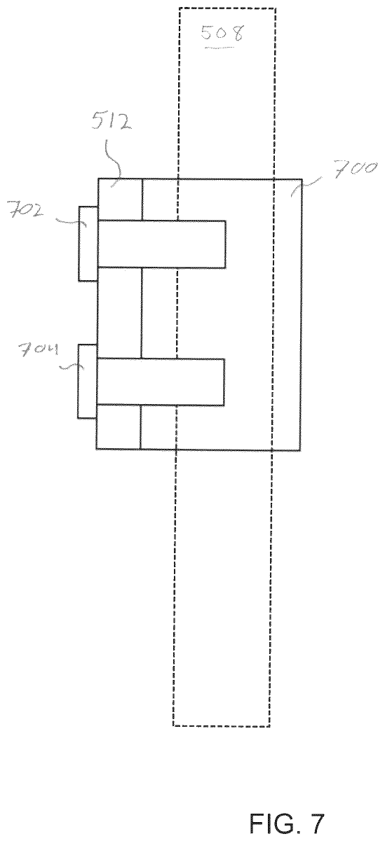

In various embodiments and with additional reference to FIG. 7, a mechanical interface module 700 is illustrated. Mechanical interface module 700 may comprise various features, materials, and components similar to those of mechanical interface modules 102 and 610. Mechanical interface module 700 includes a first fastener 702 and a second fastener 704 which couple the mechanical interface module 700 to an upright member of an ophthalmic device (e.g., upright member 508). Each of the first fastener 702 and second fastener 704 are disposed through the forehead rest (e.g. forehead rest 512) of the ophthalmic device, the mechanical interface module 700, and into an upright member of the ophthalmic device. In this regard, the first fastener 702 and second fastener 704 enable coupling between the forehead rest, the upright member, and the mechanical interface module 700.

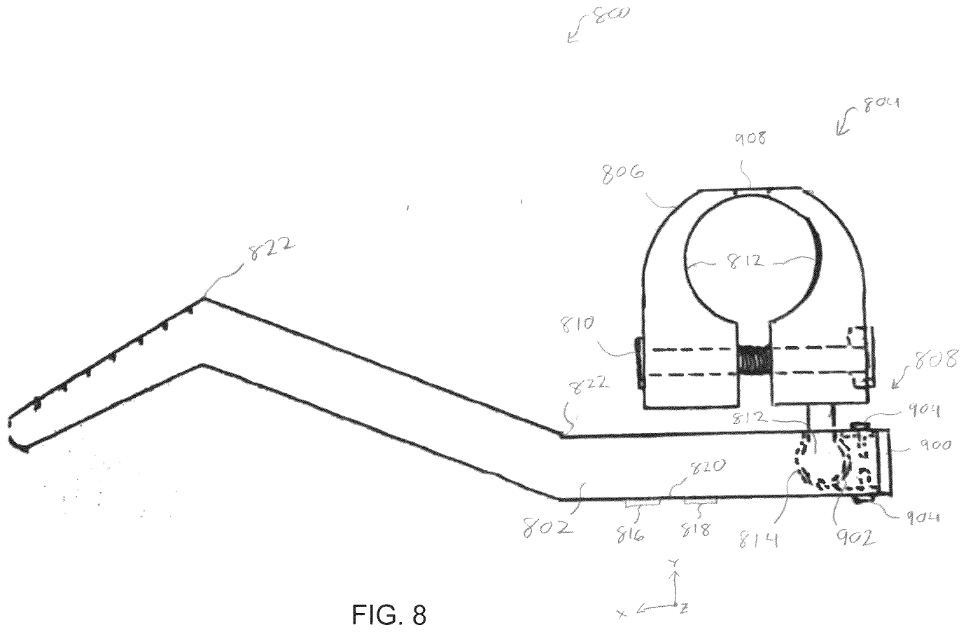

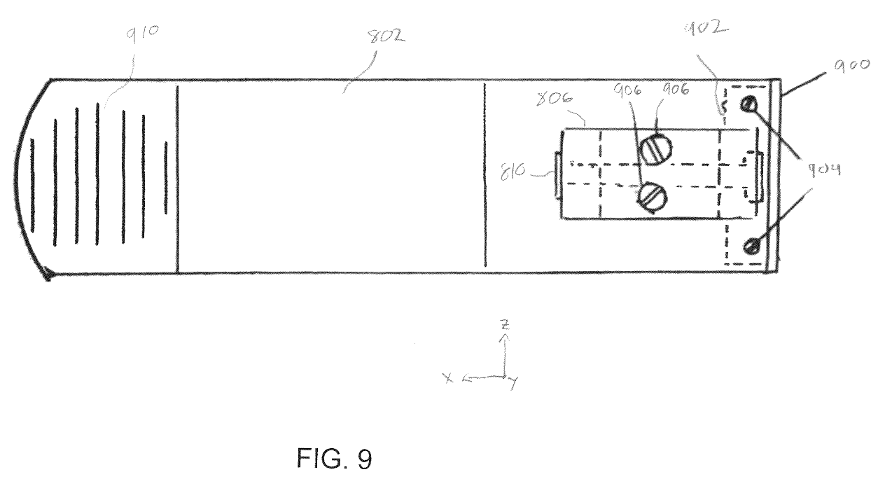

In various embodiments and with additional reference to FIGS. 8 and 9, a diagnostic test assembly 800 is illustrating showing details of a housing 802 and integrated mechanical interface module 804 with x-y-z axes included for reference. Diagnostic test assembly 800 may comprise various features, materials, and components similar to those of diagnostic test assembly 100 and 600. Housing 802 and integrated mechanical interface module 804 may comprise various features, materials, and components similar to those of housing 602 and mechanical interface modules 610 and/or 700. In various embodiments, housing 802 includes a user interface module having a button 816 and a light emitting diode 818 accessible to the user at an interface surface 820 of the housing 802. In various embodiments, housing 802 includes one or more ergonomic bends 822 which assist in positioning the housing more proximate the ears of a user when the users head is in a head fixture of a slit lamp assembly (such as, for example, head fixture 506 of slit lamp assembly 500) with the diagnostic test assembly 800 is coupled to an upright member of the slit lamp assembly.

In various embodiments, the mechanical interface module 804 comprises clamp portion 806 and a ball-and-socket joint 808. Clamp portion 806 includes a screw 810 to operate the jaws 812 and thereby affix the jaws about an upright member of an ophthalmic device such as, for example, upright member 508. In various embodiments, a ball portion 812 of the ball-and-socket joint 808 may be monolithic with clamp portion 806. The ball portion 812 may be disposed within a socket portion 814 of the ball-and-socket joint 808 which is integral with the housing 802. Ball portion 812 may be retained within the socket portion 814 by a retaining plug 900. In various embodiments, an interior surface of the retaining plug 900 may also form a part of the socket portion 814. In various embodiments, one or more retaining plug fasteners may be driven through the housing 802 into the retaining plug 900 to fix the retaining plug 900 to the housing 802.

In this regard, the ball and socket joint 808 couples the clamp portion 806 to the housing 802 and enables rotation of the housing 802 relative to the clamp portion 806 at the ball-and-socket joint 808 when the clamp portion 806 is coupled to an upright member of an ophthalmic device. In various embodiments, clamp portion 806 may include one or more forehead rest fasteners 906 which may be driven through forehead rest fastener holes 908 of the clamp portion 806 to secure a forehead rest to the mechanical interface module 804. In various embodiments, the housing may include an audio interface portion 910 comprising perforations and/or other penetrations to enable audio interface between a speaker retained within the housing proximate the audio interface portion and the user.

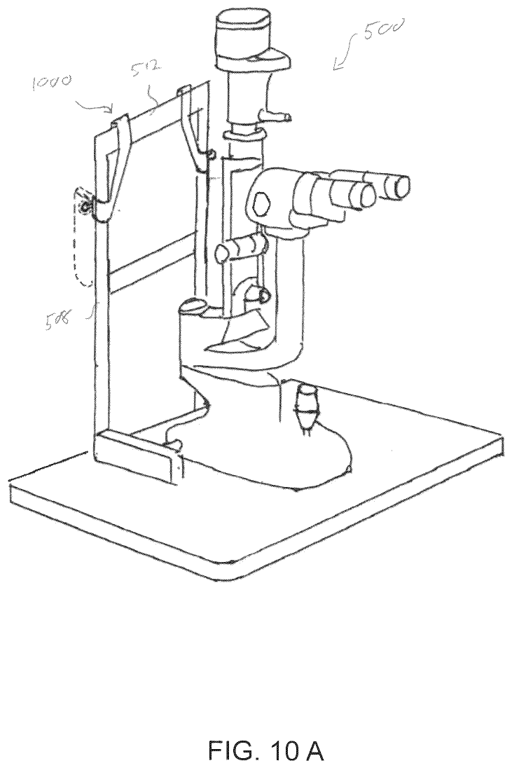

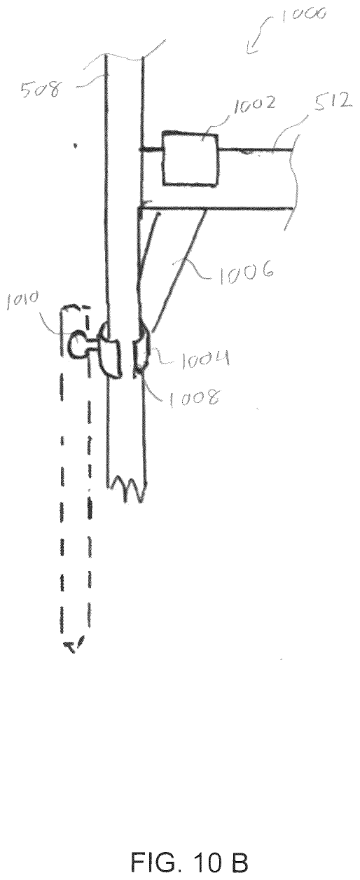

With additional reference to FIG. 10A and 10B, a mechanical interface module 1000 of a diagnostic test assembly is illustrated coupled with slit lamp assembly 500. Mechanical interface module 1000 may comprise various features, materials, and components similar to those of mechanical interface module 804. Mechanical interface module 1000 comprises a hook portion 1002 and a clamp portion 1004. The hook portion 1002 hooks over the top of the forehead rest 512 and is connected to the clamp portion 1004 by a support member 1006. Hook portion 1002 removably couples with a forehead rest 512 and the clamp portion 1004 removably couples with upright member 508. In various embodiments, the clamp portion 1004 may be a snap clamp comprising a plastic, metallic, or elastomeric material. In response to forcing opening 1008 of the clamp portion 1004 against the upright member 508, the jaws of the clamp portion 1004 may be deflected relatively outward and snap into position about the upright member 508. In this regard the clamp portion 1004 inhibits lateral translation of the mechanical interface module 1000 relative to the upright member 508. In like regard, the hook portion 1002 inhibits axial translation of the mechanical interface module 1000 relative to the upright member 508. In various embodiments the clamp portion 1004 may include a ball portion 1010 of a ball-and-socket joint.

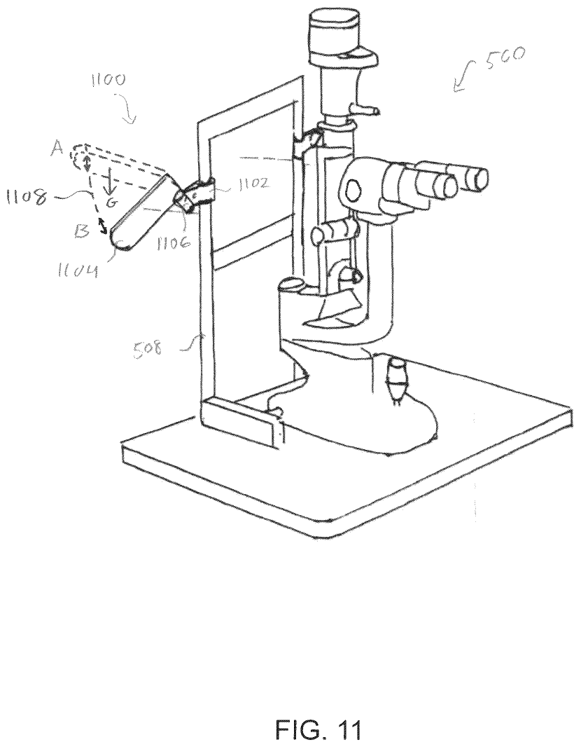

With additional reference to FIG. 11, a mechanical interface module 1100 a diagnostic test assembly is illustrated coupled with slit lamp assembly 500. Mechanical interface module 1000 may comprise various features, materials, and components similar to those of mechanical interface module 804 and 1000. Mechanical interface module 1100 includes a clamp portion 1102 coupled to a housing 1104 via an angled pivot 1106. In this regard, the housing is enabled to rotate freely about the pivot as illustrated by the dashed outline of the housing. In various embodiments, the angle of the angled pivot 1006 relative a vertical defined by the upright member 508 is selected such that, with the clamp portion 1102 coupled to upright member 508, the housing 1105 rotates to rest at a low side position B of the pivot in response to a gravitational force (arrow ‘G’) acting on the housing 1104. For example, when rotated about the pivot 1106 along the path 1108 relatively away from the low side B to position A the gravitational force G acting on the housing 1104 causes the housing to return to rest at position B.

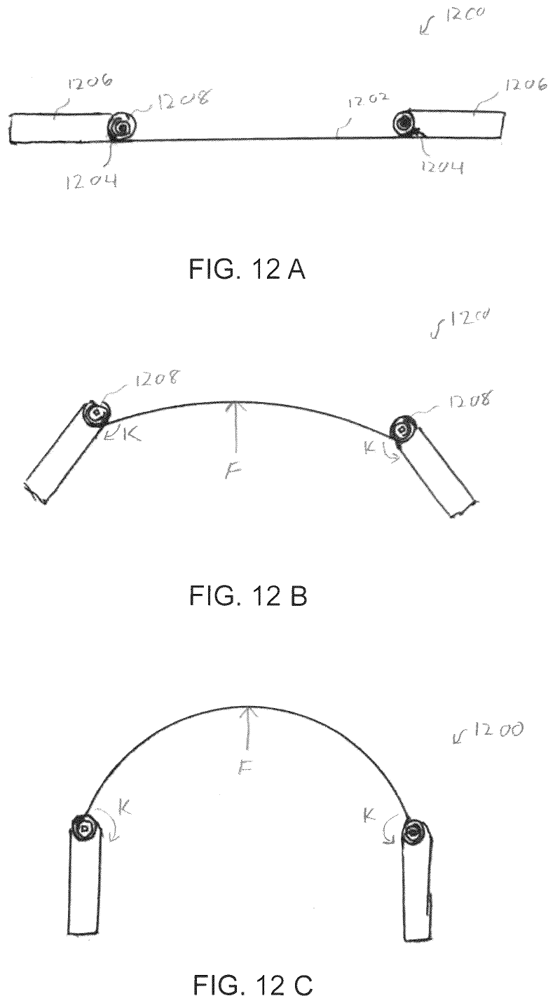

With additional reference to FIGS. 12A, 12B and 12C, a mechanical interface module 1200 of a diagnostic test assembly is illustrated as coupled with upright members of a slit lamp assembly, such as upright members 508 of slit lamp assembly 500. FIGS. 12A, 12B, and 12C show a top down view of the mechanical interface module 1200 and a housing as viewed along the axis of the upright members. Mechanical interface module 1200 may comprise various features, materials, and components similar to those of mechanical interface module 804, 1000, and 1100. Mechanical interface module 1200 further comprises a forehead rest 1202 coupled at a relatively inward face 1204 of a housing 1206. The mechanical interface module 1200 further comprises a spring 1208 coupled between the housing 1206 and the mechanical interface module 1200. As shown in FIGS. 12B and 12C, in response to a force F applied at an inward face of the forehead rest 1202, the housing begins to rotate about the upright member from its resting position or first position (FIG. 12A) through intermediate positions (FIG. 12B) to a final position or second position (FIG. 12C). In response to the rotation, spring 1208 generates a spring force K which acts to oppose the force F. In this regard, when the force F is removed from the inward face of the forehead rest 1202, the housing 1206 is acted upon by the spring force K to reverse the rotation of the housing 1206 from the second position to return to the first position.

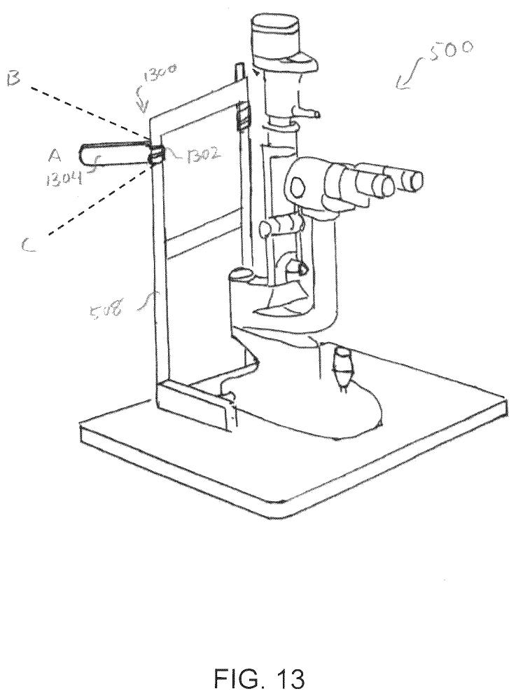

With additional reference to FIG. 13, a mechanical interface module 1300 of a diagnostic test assembly is illustrated as coupled with upright members 508 of a slit lamp assembly 500. Mechanical interface module 1300 may comprise various features, materials, and components similar to those of mechanical interface module 804, 1000, and 1100 but differs in that it includes a cam ring assembly 1302. In various embodiments, cam ring assembly 1302 enables rotation of the housing 1304 about the upright member 508 away from a resting position A in response to a clockwise rotational force toward position B or an anti-clockwise rotational force to position C. In response to removing the rotational force, the cam ring assembly 1302 causes the housing 1304 to return to the resting position A.

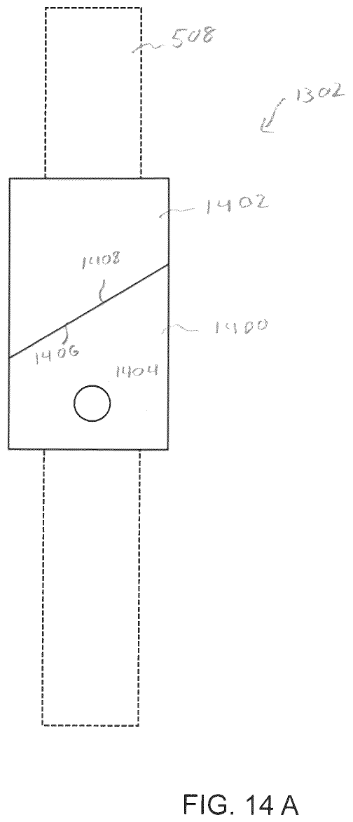

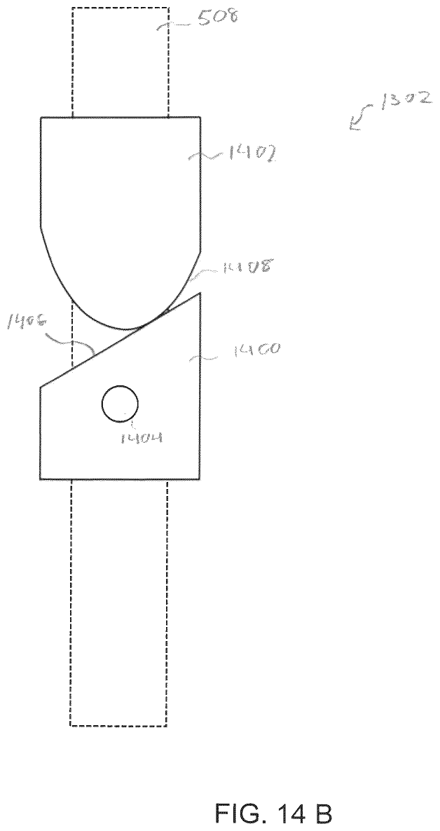

With additional reference to FIG. 14A, cam ring assembly 1302 of mechanical interface module 1300 is illustrated in the resting position A in accordance with various embodiments. Cam ring assembly 1302 comprises a lower cam ring 1400 and an upper cam ring 1402 each coupled about the upright member 508. In various embodiments, lower cam ring 1400 may be fixed to the upright member 508 by a fastener or a set screw 1404 which inhibits rotation about or translation along the upright member 508. The upper cam ring 1402 may be coupled to or monolithic with the housing 1304 and free to rotate about or translate along the upright member 508. In this regard, a portion of the housing may comprise the upper cam ring 1402. The lower cam ring 1400 has a beveled face 1406 which contacts a corresponding cam face 1408 of the upper cam ring 1402.

With additional reference to FIG. 14B, cam ring assembly 1302 of mechanical interface module 1300 is illustrated with the housing 1304 rotated to position C. As housing 1304 and upper cam ring 1402 are rotated relative to lower cam ring 1400, cam face 1408 of the upper cam ring 1402 slides along the beveled face 1406 of the lower cam ring 1400. In response to the rotation, the upper cam ring 1402 is driven upward along the beveled face 1406 causing causes axial displacement of the upper cam ring 1402 and housing 1304 along the upright member 508 relatively away from the lower cam ring 1400.

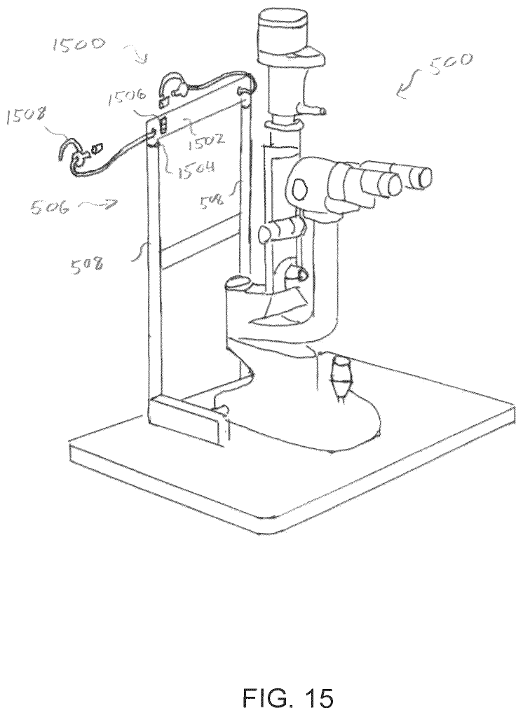

In various embodiments and with additional reference to FIG. 15, a diagnostic test assembly 1500 is illustrated. Diagnostic test assembly 1500 may comprise various features, materials, and components similar to those of diagnostic test assembly 100, 202, and 600, but differs in that is fully integrated with head fixture 506 of the slit lamp assembly 500. Diagnostic test assembly 1500 includes a housing 502 which replaces the forehead rest 512. Diagnostic test assembly 1500 includes a coupling module 1504 which couples the housing 1502 at a distal end of the upright members 508. In various embodiments, the diagnostic test assembly 1500 includes a user interface panel 1506 for the user interface module accessible at the surface of the housing 1502 and earpieces 1508 which provide an audio interface to the user.

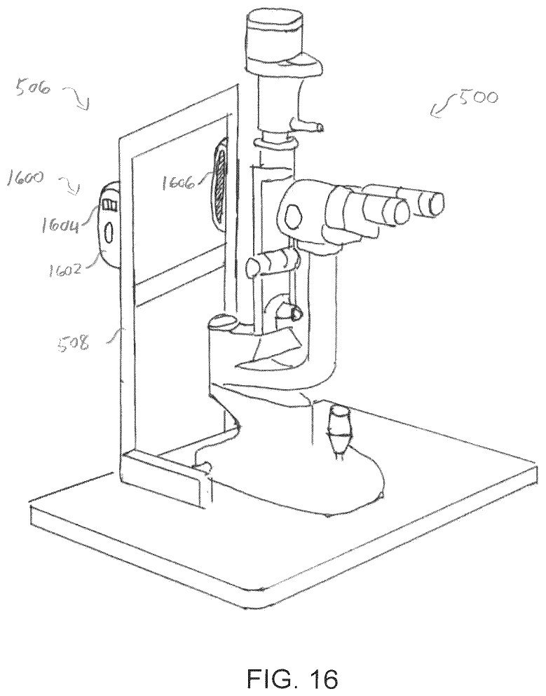

In various embodiments and with additional reference to FIG. 16, a diagnostic test assembly 1600 is illustrated. Diagnostic test assembly 1600 may comprise various features, materials, and components similar to those of diagnostic test assembly 100, 202, 600, and 1500 but differs in that is fully integrated with an upright member 508 of head fixture 506 of the slit lamp assembly 500. Diagnostic test assembly 1600 includes a housing 1602 which may be directly coupled to or integral with the upright members 508. Diagnostic test assembly 1600. In various embodiments, the diagnostic test assembly 1600 includes a user interface panel 1604 for the user interface module accessible at the surface of the housing 1600 and speakers 1606 which provide an audio interface to the user.

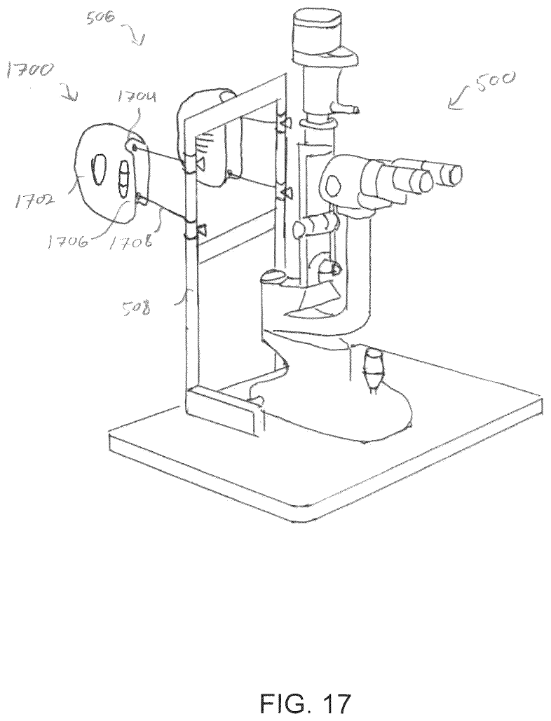

In various embodiments and with additional reference to FIG. 17, a diagnostic test assembly 1700 is illustrated. Diagnostic test assembly 1700 may comprise various features, materials, and components similar to those of diagnostic test assembly 100, 202, 600, 1500, and 1600. Diagnostic test assembly 1700 includes a housing 1700 which includes a contoured portion 1704 which matches the contour of the upright member 508 and enables the housing 1702 to slide partially around the upright member 508. Diagnostic test assembly 1700 includes a mechanical interface module having a retractable line assembly 1706 which couples the housing to the upright member 508 via retractable lines 1708. The retractable line assembly 1706 maintains tension in the retractable lines 1708 which hold the housing 1802 proximate the upright member 508. The retractable line assembly 1706 enables a user to pull the housing 1702 to relatively away from the upright member 508 to position the housing more proximate an ear of the user. In this way, the audio interface between the user and a speaker retained in the housing is enhanced when testing is conducted.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosures.

The scope of the disclosures is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods and apparatus are provided herein. In the detailed description herein, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiment

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A diagnostic test assembly for an ophthalmic device, comprising:
a housing;
an audio output module disposed within the housing;
a tone generator module disposed within the housing and in electronic communication with the audio output module;
a user interface module disposed within the housing;
a mechanical interface module integrated with the housing and configured to removably couple the housing to the ophthalmic device;
a processor disposed within the housing and in electronic communication with the user interface module and the tone generator module;
a tangible, non-transitory electronic memory in communication with the processor, the tangible, non-transitory memory having instructions stored thereon that, in response to execution by the processor, cause the processor to perform operations comprising:
receiving, by the processor, a start test command from the user interface module,
executing, by the processor, the tone generator module in response to receiving the start test command,
wherein, in response to execution by the processor, the tone generator module generates a discrete series of pure tone sine waves and feeds the discrete series of pure tone sine waves to the audio output module for further processing,
wherein, in response to receiving the discrete series of pure tone sine waves, the audio output module modulates the output level of each tone of the of the discrete series of pure tone sine waves across a discrete series of audio output levels.

2. The diagnostic test assembly of claim 1, wherein the discrete series of pure tone sine waves consists of tones at 500 Hz, 1000 Hz, 2000 Hz, and 4000 Hz.

3. The diagnostic test assembly of claim 2, the modulation of the audio output level begins at an output level of 50 dB and decreases in intensity thereafter to 10 dB or less.

4. The diagnostic test assembly of claim 3, wherein the modulation of the audio output level decreases by decrements of at least 5 dB to 10 dB.

5. The diagnostic test assembly of claim 1, wherein the user interface module comprises at least one of a button or a light emitting diode.

6. The diagnostic test assembly of claim 1, wherein the mechanical interface module comprises clamp portion and a ball-and-socket joint, wherein the ball-and-socket joint couples the clamp portion to the housing and enables rotation of the housing relative to the clamp portion at the ball-and-socket joint.

7. The diagnostic test assembly of claim 6, wherein a ball portion of the ball-and-socket joint is disposed within the housing and retained therein by a retaining plug.

8. The diagnostic test assembly of claim 1, wherein the mechanical interface module further comprises a hook portion and a clamp portion, wherein the hook portion removably couples with a forehead rest of the ophthalmic device and the clamp portion removably couples with an upright member of the ophthalmic device.

9. The diagnostic test assembly of claim 1, wherein the mechanical interface module further comprises a forehead rest coupled to the housing and a spring coupled between the housing and the mechanical interface module.

10. The diagnostic test assembly of claim 9, wherein with the mechanical interface module coupled to an upright member of the ophthalmic device, to in response to a force applied at an inward face of the forehead rest, the housing rotates about the upright member from a first position to a second position and generates a spring force via the spring.

11. The diagnostic test assembly of claim 9, wherein in response to removing the force applied at the inward face of the forehead rest, the spring force acts to rotate the housing from the second position to the first position.

12. The diagnostic test assembly of claim 1, wherein the mechanical interface module further comprises a first fastener and a second fastener, wherein each of the first fastener and the second fastener are disposed through a forehead rest of the ophthalmic device, the mechanical interface module, and into an upright member of the ophthalmic device to enable coupling therebetween.

13. The diagnostic test assembly of claim 1, wherein the mechanical interface module further comprises a clamp portion coupled to the housing via an angled pivot, wherein the housing is enabled to rotate freely about the pivot, and wherein with the clamp portion coupled to an upright member of the ophthalmic device, in response to a gravitational force action on the housing, the housing rotates to rest at a low side of the pivot.

14. The diagnostic test assembly of claim 1, wherein the mechanical interface module further comprises a cam ring having a beveled face contacting a portion of the housing such that when coupled about an upright member of the ophthalmic device a rotation of the housing relative to the cam ring causes axial displacement of the housing along the upright member relatively away from the cam ring.

15. A diagnostic test assembly for an ophthalmic device, comprising:
a mobile device including a display and an audio output device;
a mechanical interface module configured to removably couple the mobile device to the ophthalmic device;
a processor;
a tangible, non-transitory electronic memory in electronic communication with the processor, and
a set of computer readable code on the non-transitory electronic memory, including:
a user interface (UI) module executable to display system information and receive user inputs;
a tone generator module executable by the processor to generate pure tone sine waves between 20 Hz and 10,000 Hz;
an audio output module executable by the processor to generate audio output via the audio output device;
wherein, in response to execution by the processor, the UI module awaits to receive a start test command,
wherein, in response to receiving the start test command, the UI module calls the processor to execute the tone generator module,
wherein, in response to execution by the processor, the tone generator module generates a discrete series of pure tone sine waves and feeds the discrete series of pure tone sine waves to the audio output module for further processing,
wherein, in response to receiving the discrete series of pure tone sine waves, the audio output module controls the audio output device to output, sequentially, each tone of the of the discrete series of pure tone sine waves across a discrete series of audio output levels.

16. The diagnostic test assembly of claim 15, further comprising a native application executable on the mobile device, wherein the native application includes the UI module, the tone generator module, and the audio output module.

17. The diagnostic test assembly of claim 16, wherein the discrete series of pure tone sine waves consists of tones at 500 Hz, 1000 Hz, 2000 Hz, and 4000 Hz.

18. The diagnostic test assembly of claim 16, wherein the discrete series of audio output levels begins at an output level of 50 dB and decreases in intensity thereafter to 10 dB or less.

19. The diagnostic test assembly of claim 18, wherein the discrete series of audio output levels steps down by decrements of at least 5 dB to 10 dB.

20. The diagnostic test assembly of claim 15, wherein the mechanical interface module includes a clamp portion, a mobile device mounting portion, and an arm portion, wherein the clamp portion and the mobile device mounting portion are coupled at distal ends of the arm portion.
